(19) United States
(12) Patent Application Publication
(10) Pub. No.: US 2009/0281154 A1
(43) Pub. Date: Nov. 12, 2009

(54) Ophthalmic Compositions for Treating Ocular Hypertension

(76) Inventors: James B. Doherty, Montvale, NJ (US); Swaminathan R. Natarajan, Scotch Plains, NJ (US); Dong-Ming Shen, Edison, NJ (US); Fengqi Zhang, Edison, NJ (US)

Correspondence Address: MERCK AND CO., INC, P O BOX 2000, RAHWAY, NJ, 07065-0907, US

(21) Appl. No.: 12/308,164

(22) PCT Filed: Jun. 7, 2007
(86) PCT No.: PCT/US07/13528

§ 371 Date: Dec. 8, 2008

Related U.S. Application Data
(60) Provisional application No. 60/812,839, filed on Jun. 12, 2006.

(51) Int. Cl. A61K 31/426; C07C 233/11; A61K 31/165; C07D 277/38; A61P 27/06; A61P 27/02

(52) U.S. Cl. 514/371; 564/169; 514/621; 548/195

(57) ABSTRACT

This invention relates to the use of potent potassium channel blockers or a formulation thereof in the treatment of glaucoma and other conditions which leads to elevated intraoccular pressure in the eye of a patient. This invention also relates to the use of such compounds to provide a neuroprotective effect to the eye of mammalian species, particularly humans.

### BACKGROUND OF THE INVENTION

Glaucoma is a degenerative disease of the eye wherein the intraocular pressure is too high to permit normal eye function. As a result, damage may occur to the optic nerve head and result in irreversible loss of visual function. If untreated, glaucoma may eventually lead to blindness. Ocular hypertension, i.e., the condition of elevated intraocular pressure without optic nerve head damage or characteristic glaucomatous visual field defects, is now believed by the majority of ophthalmologists to represent merely the earliest phase in the onset of glaucoma.

There are several current therapies for treating glaucoma and elevated intraocular pressure (e.g., pilocarpine, beta blockers (e.g., timolol), carbonic anhydrase inhibitors (e.g., dorzolamide, brinzolamide) and prostaglandins (e.g., latanoprost), but the efficacy and the side effect profiles of these agents are not ideal. Recently potassium channel blockers were found to reduce intraocular pressure in the eye and therefore provide yet one more approach to the treatment of ocular hypertension and the degenerative ocular conditions related thereto. Blockage of potassium channels can diminish fluid secretion, and under some circumstances, increase smooth muscle contraction and would be expected to lower IOP and have neuroprotective effects in the eye. (see U.S. Pat. Nos. 5,573,758 and 5,925,342; Moore, et al., Invest. Opthalmol. Vis. Sci 38, 1997; WO 89/10757, WO94/28900, and WO 96/33719).

### SUMMARY OF THE INVENTION

This invention relates to the use of potent naphthalene derivatives as potassium channel blockers or a formulation thereof in the treatment of glaucoma and other conditions which are related to elevated intraocular pressure in the eye of a patient. This invention also relates to the use of such compounds to provide a neuroprotective-effect to the eye of mammalian species, particularly humans. More particularly this invention relates to the treatment of glaucoma and/or ocular hypertension (elevated intraocular pressure) using novel naphthalene derivatives having the structural formula I:

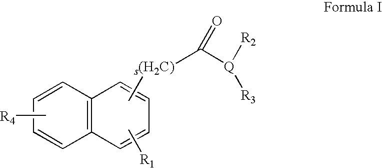

or a pharmaceutically acceptable salt, ester including phosphate, enantiomer, diastereomer or mixture thereof:
wherein,
R and R^{y} independently represent hydrogen, or C₁₋₆ alkyl;
R₁ represents hydrogen or C₁₋₆ alkyl, CF₃, (CH₂)ₙC₃₋₁₀ cycloalkyl, (CH₂)ₙC₆₋₁₀ aryl, —(CH₂)ₙC₅₋₁₀ heteroaryl, C₁₋₆ alkoxy, OH, COR^{c}, said alkyl, cycloalkyl, aryl, heteroaryl, and alkoxy optionally substituted with 1-3 groups selected from R^{b};
Q represents N, CR^{y}, or O, wherein R₂ is absent when Q is O;
R₂ represents hydrogen, C₁₋₁₀ alkyl, C₂₋₁₀ hydroxylalkyl, C₁₋₆ alkyl SR, —(CH₂)ₙO(CH₂)ₘOR, (CH₂)ₘOR, —(CH₂)ₙ(CHR₇)_{S}(CH₂)ₘC₁₋₆ alkoxy, —(CH₂)ₙ(CHR₇)_{S}(CH₂)ₘC₃₋₈ cycloalkyl, —(CH₂)ₙ(CHR₇)_{S}(CH₂)ₘC₃₋₁₀ heterocyclyl, —(CH₂)ₙC₅₋₁₀ heteroaryl, —N(R)₂, —COOR, or —(CH₂)ₙ(CHR₇)_{S}(CH₂)ₘC₆₋₁₀ aryl, said alkyl, cycloalkyl, heterocyclyl, aryl or heteroaryl optionally substituted with 1-3 groups selected from R^{a};
R₃ represents hydrogen, C₁₋₁₀ alkyl, C₂₋₆ alkenyl, —(CH₂)ₙ(CHR₇)_{S}(CH₂)ₘC₃₋₈ cycloalkyl, —(CH₂)ₙ(CHR₇)_{S}(CH₂)ₘC₃₋₁₀ heterocyclyl, —(CH₂)ₙ(CHR₇)_{S}(CH₂)ₘC₅₋₁₀ heteroaryl, —(CH₂)ₙ(CHR₇)_{S}(CH₂)ₘCOOR, —(CH₂)ₙ(CHR₇)_{S}(CH₂)ₘC₆₋₁₀ aryl, —(CH₂)ₙ(CHR₇)_{S}(CH₂)ₘNHR₈, —(CH₂)ₙ(CHR₇)_{S}(CH₂)ₘN(R)₂, —(CH₂)ₙ(CHR₇)_{S}(CH₂)ₘN(R₈)₂, —(CH₂)ₙ(CHR₇)_{S}(CH₂)ₘNHCOOR, —(CH₂)ₙ(CHR₇)_{S}(CH₂)ₘN(R₈)CO₂R, —(CH₂)ₙ(CHR₇)_{S}(CH₂)ₘN(R₈)COR, —(CH₂)ₙ(CHR₇)_{S}(CH₂)ₘNHCOR, —(CH₂)ₙ(CHR₇)_{S}(CH₂)ₘCONH(R₈), aryl, —(CH₂)ₙ(CHR₇)_{S}(CH₂)ₘOR, —(CH₂)ₙC(R₇)₂(CH₂)ₘOR, CF₃, (CH₂)ₙ(CHR₇)_{S}(CH₂)ₘSO₂R, —(CH₂)ₙ(CHR₇)_{S}(CH₂)ₘSO₂N(R)₂, —(CH₂)ₙ(CHR₇)_{S}(CH₂)ₘCON(R)₂, —(CH₂)ₙ(CHR₇)_{S}(CH₂)ₘCONHC(R)₃, —(CH₂)ₙCONHC(R)₂CO₂R, —(CH₂)ₙ(CHR₇)_{S}(CH₂)ₘCOR₈, nitro, cyano or halogen, said alkyl, cycloalkyl, alkoxy, heterocyclyl, aryl or heteroaryl optionally substituted with 1-3 groups of R^{a};
or, when Q is N, R₂ and R₃ taken together with the intervening N atom form a 4-10 membered heterocyclic ring optionally interrupted by 1-2 atoms of O, S, C(O) or NR, and optionally having 1-4 double bonds, and optionally substituted by 1-3 groups selected from R^{a};
or, when Q equals CR^{y}, R₂ and R₃ taken together with the intervening CR^{y} form a 4-10 membered carbocyclic or heterocyclic aromatic ring or fused ring optionally interrupted by 1-2 atoms of O, S, C(O) or NR, and optionally having 1-5 double bonds, and optionally substituted by 1-3 groups selected from R^{a};
R₄ represents hydrogen, C₁₋₆ alkoxy, halogen, cyano, OH, C₁₋₆ alkyl, COOR, SO₃H, C₁₋₆ alkylcarbonyl, S(O)_{q}R^{y}, —O(CH₂)ₙN(R)₂, —O(CH₂)ₙCO₂R, —OPO(OH)₂, CF₃, —N(R)₂, nitro, or C₁₋₆ alkylamino;
R₇ represents hydrogen, C₁₋₆ alkyl, —(CH₂)ₙCOOR or —(CH₂)ₙN(R)₂,
R₈ represents —(CH₂)ₙC₃₋₈ cycloalkyl, —(CH₂)_{n 3}-10 heterocyclyl, C₁₋₆ alkoxy or —(CH₂)ₙC₅₋₁₀ heteroaryl, —(CH₂)ₙC₆₋₁₀ aryl said heterocyclyl, cycloalkyl, aryl or heteroaryl optionally substituted with 1-3 groups selected from R^{a};
R^{a} represents F, Cl, Br, I, CF₃, N(R)₂, NO₂, CN, —(CH₂)ₙCOR₈, —(CH₂)ₙCONHR₈, —(CH₂)ₙCON(R₈)₂, —O(CH₂)ₙCOOR, —NH(CH₂)ₙOR, —COOR, —OCF₃, —O—, —NHCOR, —SO₂R, —SO₂NR₂, —SR, (C₁-C₆ alkyl)O—, —(CH₂)ₙO(CH₂)ₘOR, —(CH₂)ₙC₁₋₆ alkoxy, (aryl)O—, —OH, (C₁-C₆ alkyl)S(O)ₘ—, H₂N—C(NH)—, (C₁-C₆ alkyl)C(O)—, (C₁-C₆ alkyl)OC(O)NH—, —(C₁-C₆ alkyl)NR_{w}(CH₂)ₙC₃₋₁₀ heterocyclyl-R_{w}, —(C₁-C₆ alkyl)O(CH₂)ₙC₃₋₁₀ heterocyclyl-R_{w}, —(C₁-C₆ alkyl)S(CH₂)ₙC₃₋₁₀ heterocyclyl-R_{w}, —(C₁-C₆ alkyl)-C₃₋₁₀ heterocyclyl-R_{w}, —(CH₂)ₙ—K—C(═K)N(R)₂, —(C₂₋₆ alkenyl)NR_{w}(CH₂)ₙC₃₋₁₀ heterocyclyl-R_{w}, —(C₂₋₆ alkenyl)O(CH₂)ₙC₃₋₁₀ heterocyclyl-R_{w}, —(C₂₋₆ alkenyl)S(CH₂)ₙC₃₋₁₀ heterocyclyl-R_{w}, —(C₂₋₆ alkenyl)-C₃₋₁₀ heterocyclyl-R_{w}, —(C₂₋₆ alkenyl)-K—C(═K)N(R)₂, —(CH₂)ₙSO₂R, —(CH₂)ₙSO₃H, —(CH₂)ₙPO(OR)₂, —(CH₂)ₙOPO(OR)₂, cyclohexyl, cyclopentyl, morpholinyl, piperidyl, pyrrolidinyl, thiophenyl, phenyl, pyridyl, imidazolyl, oxazolyl, isoxazolyl, thiazolyl, thienyl, furyl, isothiazolyl, C₂₋₆ alkenyl, and C₁-C₁₀ alkyl, said alkyl, alkenyl, alkoxy, phenyl, pyridyl, imidazolyl, oxazolyl, isoxazolyl, thiazolyl, thienyl, furyl, and isothiazolyl optionally substituted with 1-3 groups selected from C₁-C₆ alkyl, and COOR;
K independently represents CH, CH₂ or NH;
R_{w} represents H, C₁₋₆ alkyl, —C(O)C₁₋₆ alkyl, —C(O)OC₁₋₆ alkyl, —SO₂N(R)₂, —SO₂C₁₋₆ alkyl, —SO₂C₆₋₁₀ aryl, NO₂, CN or —C(O)N(R)₂;
R^{b} represents C₁₋₆ alkyl, —COOR, —SO₃R, CN, (CH₂)ₙOR, C(O)O(CH₂)ₙC(O)R, —OPO(OH)₂, —(CH₂)ₙC₆₋₁₀aryl, or —(CH₂)ₙC₅₋₁₀ heteroaryl;
R^{c} represents hydrogen, C₁₋₆ alkyl, or —(CH₂)ₙC₆₋₁₀ aryl;
m is 0-3;
n is 0-3;
q is 0-2; and
s is 0-2.

This and other aspects of the invention will be realized upon inspection of the invention as a whole.

### DETAILED DESCRIPTION OF THE INVENTION

The present invention is directed to novel potassium channel blockers of Formula I. It also relates to a method for decreasing elevated intraocular pressure or treating glaucoma by administration, preferably topical or intra-camaral administration, of a composition containing a potassium channel blocker of Formula I described hereinabove and a pharmaceutically acceptable carrier. This invention also relates to the use of the compounds of Formula I for the manufacture of a medicament in the treatment of ocular diseases such as glaucoma, ocular hypertension, macular degeneration and the like.

An embodiment of this invention is realized when Q is N and all other variables are as originally described.

Another embodiment of this invention is realized when Q is CH or CCH₃ and all other variables are as originally described.

Still another embodiment R_{w} is selected from H, C₁₋₆ alkyl, —C(O)C₁₋₆ alkyl and —C(O)N(R)₂.

Another embodiment of this invention is realized when QR₂R₃ is a dialkylamine or hydroxylamine and all other variables are as originally described.

Still another embodiment of this invention is realized when R₁ is C₁₋₆alkyl, and QR₂R₃ is a dialkylamine or hydroxyldialkylamine and all other variables are as originally described.

Still another embodiment of this invention is realized when R₁ is —C(O)Rc, and QR₂R₃ is a dialkylamine or hydroxyldialkylamine and all other variables are as originally described. A subembodiment of this invention is realized when R^{c} is phenyl optionally substituted with 1 to 3 groups of R^{a}

Yet another embodiment of this invention is realized when R₇ is hydrogen or C₁₋₆ alkyl, and all other variables are as originally described.

Another embodiment of the instant invention is realized when R^{a} is selected from F, Cl, Br, I, CF₃, N(R)₂, NO₂, CN, —CONHR₈, —CON(R₈)₂, —O(CH₂)ₙCOOR, —NH(CH₂)ₙOR, —COOR, —OCF₃, —NHCOR, —SO₂R, —SO₂NR₂, —SR, (C₁-C₆ alkyl)O—, —(CH₂)ₙO(CH₂)ₘOR, —(CH₂)ₙC₁₋₆ alkoxy, (aryl)O—, —OH, (C₁-C₆ alkyl)S(O)ₘ—, H₂N—C(NH)—, (C₁-C₆ alkyl)C(O)—, (C₁-C₆ alkyl)OC(O)NH—, —(C₁-C₆ alkyl)NR_{w}(CH₂)ₙC₃₋₁₀ heterocyclyl-R_{w}, —(CH₂)ₙ—K—C(═K)N(R)₂, —(C₂₋₆ alkenyl)NR_{w}(CH₂)ₙC₃₋₁₀ heterocyclyl-R_{w}, —(C₂₋₆ alkenyl)-K—C(═K)N(R)₂, —(CH₂)ₙSO₂R, —(CH₂)ₙSO₃H, —(CH₂)ₙPO(OR)₂, —(CH₂)ₙOPO(OR)₂, C₂₋₆ alkenyl, and C₁-C₁₀ alkyl, said alkyl and alkenyl, optionally substituted with 1-3 groups selected from C₁-C₆ alkyl, and COOR;

Still another embodiment of this invention is realized when Q is N, and R₂ and R₃ are taken together with the intervening N atom form a 4-10 membered heterocyclic carbon ring optionally interrupted by 1-2 atoms of O, S, C(O) or NR, and optionally having 1-4 double bonds, and optionally substituted by 1-3 groups selected from R^{a}. Examples of said heterocyclic groups are:

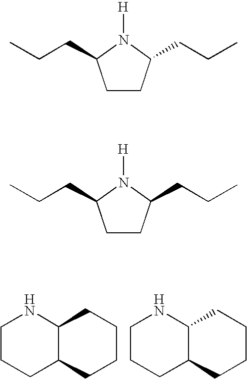

and the like.

Still another embodiment of this invention is realized when Q equals CR^{y}, and R₂ and R₃ taken together with the intervening CR^{y} form a 4-10 membered carbocyclic or heterocyclic aromatic ring or fused ring optionally interrupted by 1-2 atoms of O, S, C(O) or NR, and optionally having 1-5 double bonds, and optionally substituted by 1-3 groups selected from R^{a}. Examples of said groups are phenyl, pyridinyl, adamantyl, [1.1.1]bicyclopentyl, and the like.

Another embodiment of this invention is realized by structural formula II:

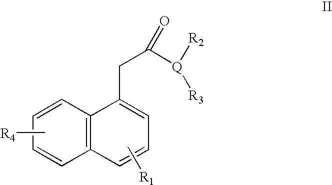

or a pharmaceutically acceptable salt, enantiomer, diastereomer or mixture thereof:
wherein,

### Q is N;

R₁ represents hydrogen or COR^{c}, C₁₋₆ alkyl, (CH₂)ₙC₃₋₁₀ cycloalkyl, (CH₂)ₙC₆₋₁₀ aryl, —(CH₂)ₙC₅₋₁₀ heterocyclyl, C₁₋₆ alkoxy, said alkyl, cycloalkyl, aryl, heterocyclyl and alkoxy optionally substituted with 1-3 groups selected from R^{b};
R₂ represents hydrogen, C₁₋₁₀ alkyl, C₂₋₁₀ hydroxylalkyl, (CH₂)ₘOR, —(CH₂)ₙ(CHR₇)_{S}(CH₂)ₘC₁₋₆ alkoxy, —(CH₂)ₙ(CHR₇)_{S}(CH₂)ₘC₃₋₈ cycloalkyl, —(CH₂)ₙ(CHR₇)_{S}(CH₂)ₘC₃₋₁₀ heterocyclyl, —(CH₂)ₙC₅₋₁₀ heteroaryl, or —(CH₂)ₙ(CHR₇)S(CH₂)ₘC₆₋₁₀ aryl, said alkyl, cycloalkyl, heterocyclyl, aryl or heteroaryl optionally substituted with 1-3 groups selected from R^{a};
R₃ represents hydrogen, C₁₋₁₀ alkyl, —(CH₂)ₙ(CHR₇)_{S}(CH₂)ₘC₃₋₈ cycloalkyl, —(CH₂)ₙ(CHR₇)_{S}(CH₂)ₘC₃₋₁₀ heterocyclyl, —(CH₂)ₙ(CHR₇)_{S}(CH₂)ₘC₅₋₁₀ heteroaryl, or —(CH₂)ₙ(CHR₇)_{S}(CH₂)ₘC₆₋₁₀ aryl, —(CH₂)ₙOPO(OR)₂, said alkyl, cycloalkyl, alkoxy, heterocyclyl, aryl or heteroaryl optionally substituted with 1-3 groups of R^{a}; and all other variables are as described herein.

A sub-embodiment of the compounds of formula II is realized when R₁ is CO—C₁₋₆ alkyl, optionally substituted with 1 to 3 groups of R^{b}. Examples of C₁₋₆ alkyls are t-butyl, ethyl, isopropyl, methyl and the like. Another sub-embodiment of the compounds of formula II is realized when R₁ is hydrogen. Still another sub-embodiment of the compounds of formula II is realized when R₁ is COR^{c} and R^{c} is (CH₂)ₙC₆₋₁₀ aryl, optionally substituted with 1 to 3 groups of R^{b}. Yet another sub-embodiment of the compounds of formula II is realized when R₁ is (CH₂)ₙC₃₋₁₀ cycloalkyl, optionally substituted with 1 to 3 groups of R^{b}.

Another sub-embodiment of the compounds of formula II is realized when R₂ and R₃ are independently C₁₋₁₀ alkyl, —(CH₂)ₙ(CHR₇)_{S}(CH₂)ₘC₆₋₁₀ aryl, (CH₂)ₙ(CHR₇)_{S}(CH₂)ₘC₃₋₁₀ heterocyclyl, said alkyl, heterocyclyl, aryl optionally substituted with 1-3 groups selected from R^{a}.

Another sub-embodiment of the compounds of formula II is realized when R₂ and R₃ are independently hydrogen, C₁₋₁₀ alkyl, said alkyl, optionally substituted with 1-3 groups selected from R^{a}.

Still another embodiment of this invention is realized with a compound of structural formula III:

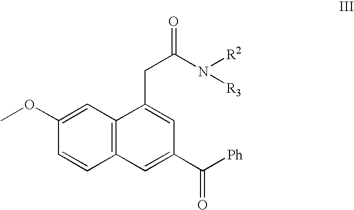

Another sub-embodiment of the compounds of formula II is realized when R₂ and R₃ are independently C₁₋₁₀ alkyl, (CH₂)ₙC₃₋₁₀ cycloalkyl, —(CH₂)ₙ(CHR₇)_{S}(CH₂)ₘC₆₋₁₀ aryl, (CH₂)ₙ(CHR₇)_{S}(CH₂)ₘC₃₋₁₀ heterocyclyl, said alkyl, cycloalkyl, heterocyclyl, and aryl optionally substituted with 1-3 groups selected from R^{a}.

Another sub-embodiment of the compounds of formula II is realized when R₂ and R₃ are independently hydrogen, C₁₋₁₀ alkyl, said alkyl, optionally substituted with 1-3 groups selected from R^{a}. A sub-embodiment of this invention is realized when both R₂ and R₃ are C₁₋₁₀ alkyl optionally substituted with 1-3 groups of R^{a}.

Examples of compounds to be used in this invention are:
2-(3-Benzoyl-7-methoxy-1-naphthyl)-N,N-dipropylacetamide;
2-(3-Benzoyl-7-methoxy-1-naphthyl)-N,N-dibutylacetamide;
2-(3-Benzoyl-7-methoxy-1-naphthyl)-N-(cyclopropylmethyl)-N-propylacetamide;
2-(3-Benzoyl-7-methoxy-1-naphthyl)-N-(cyclohexyl)-N-ethylacetamide;
2-(3-Benzoyl-7-methoxy-1-naphthyl)-N-ethyl-N-butylacetamide;
2-(3-Benzoyl-7-methoxy-1-naphthyl)-N,N-diisobutylacetamide;
2-(3-Benzoyl-7-methoxy-1-naphthyl)-N,N-bis(3-methylbutyl)acetamide;
2-(3-Benzoyl-7-methoxy-1-naphthyl)-N-ethyl-N-(3-methylbutyl)acetamide;
2-(3-Benzoyl-7-methoxy-1-naphthyl)-N-(3,3-dimethylbutyl)-N-ethylacetamide;
2-[7-Methoxy-3-(3-methylbutanoyl)-1-naphthyl]-N,N-bis(3-methylbutyl)acetamide;
2-(4-Benzoyl-7-methoxy-1-naphthyl)-N,N-bis(3-methylbutyl)acetamide;
2-(1-Benzoyl-7-methoxy-2-naphthyl)-N,N-dibutylacetamide;
2-(2-Benzoyl-7-methoxy-1-naphthyl)-N,N-dibutylacetamide;
4-Benzoyl-N-ethyl-7-methoxy-N-1,3-thiazol-2-yl-2-naphthamide;
2-(4-Benzoyl-7-methoxy-2-naphthyl)-N-ethyl-N-1,3-thiazol-2-ylacetamide;
   or a pharmaceutically acceptable salt, ester, including phosphate, enantiomer, diastereomer or mixture thereof.

This invention is described herein in detail using the terms defined below unless otherwise specified.

The compounds of the present invention may have asymmetric centers, chiral axes and chiral planes, and occur as racemates, racemic mixtures, and as individual diastereomers, with all possible isomers, including optical isomers, being included in the present invention. (See E. L. Eliel and S. H. Wilen Stereochemistry of Carbon Compounds (John Wiley and Sons, New York 1994), in particular pages 1119-1190)

When any variable (e.g. aryl, heterocycle, R₁, R₄ etc.) occurs more than one time in any constituent, its definition on each occurrence is independent at every other occurrence. Also, combinations of substituents/or variables are permissible only if such combinations result in stable compounds.

When R^{a} is —O— and attached to a carbon it is referred to as a carbonyl group and when it is attached to a nitrogen (e.g., nitrogen atom on a pyridyl group) or sulfur atom it is referred to a N-oxide and sulfoxide group, respectively.

The term “alkyl” refers to a monovalent alkane (hydrocarbon) derived radical containing from 1 to 10 carbon atoms unless otherwise defined. It may be straight, branched or cyclic. Preferred alkyl groups include methyl, ethyl, propyl, isopropyl, butyl, t-butyl, cyclopropyl cyclopentyl and cyclohexyl. When the alkyl group is said to be substituted with an alkyl group, this is used interchangeably with “branched alkyl group”.

Cycloalkyl is a specie of alkyl containing from 3 to 15 carbon atoms, unless otherwise defined, without alternating or resonating double bonds between carbon atoms. It may contain from 1 to 4 rings, which can be fused. Examples of such cycloalkyl elements include, but are not limited to, cyclopropyl, cyclobutyl, cyclopentyl, cyclohexyl and cycloheptyl.

Alkenyl is C₂-C₆ alkenyl.

Alkoxy refers to an alkyl group of indicated number of carbon atoms attached through an oxygen bridge, with the alkyl group optionally substituted as described herein. Said groups are those groups of the designated length in either a straight or branched configuration and if two or more carbon atoms in length, they may include a double or a triple bond. Exemplary of such alkoxy groups are methoxy, ethoxy, propoxy, isopropoxy, butoxy, isobutoxy, tertiary butoxy, pentoxy, isopentoxy, hexoxy, isohexoxy allyloxy, propargyloxy, and the like.

Halogen (halo) refers to chlorine, fluorine, iodine or bromine.

Aryl refers to aromatic rings e.g., phenyl, substituted phenyl and the like, as well as rings which are fused, e.g., naphthyl, phenanthrenyl and the like. An aryl group thus contains at least one ring having at least 6 atoms, with up to five such rings being present, containing up to 22 atoms therein, with alternating (resonating) double bonds between adjacent carbon atoms or suitable heteroatoms. Examples of aryl groups are phenyl, naphthyl, tetrahydronaphthyl, indanyl, biphenyl, phenanthryl, anthryl or acenaphthyl and phenanthrenyl, preferably phenyl, naphthyl or phenanthrenyl. Aryl groups may likewise be substituted as defined. Preferred substituted aryls include phenyl and naphthyl.

The term heterocyclyl or heterocyclic, as used herein, represents a stable 3- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. A fused heterocyclic ring system may include carbocyclic rings and need include only one heterocyclic ring. The term heterocycle or heterocyclic includes heteroaryl moieties. Examples of such heterocyclic elements include, but are not limited to, azepinyl, benzimidazolyl, benzisoxazolyl, benzofulrazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, dihydropyrrolyl, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperidinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl. Preferably, heterocycle is selected from 2-azepinonyl, benzimidazolyl, 2-diazapinonyl, dihydroimidazolyl, dihydropyrrolyl, imidazolyl, 2-imidazolidinonyl, indolyl, isoquinolinyl, morpholinyl, piperidyl, piperazinyl, pyridyl, pyrrolidinyl, 2-piperidinonyl, 2-pyrimidinonyl, 2-pyrollidinonyl, quinolinyl, tetrahydrofuryl, tetrahydroisoquinolinyl, and thienyl.

The term “heteroatom” means O, S or N, selected on an independent basis.

The term “heteroaryl” refers to a monocyclic aromatic hydrocarbon group having 5 or 6 ring atoms, or a bicyclic aromatic group having 8 to 10 atoms, containing at least one heteroatom, O, S or N, in which a carbon or nitrogen atom is the point of attachment, and in which one or two additional carbon atoms is optionally replaced by a heteroatom selected from O or S, and in which from 1 to 3 additional carbon atoms are optionally replaced by nitrogen heteroatoms, said heteroaryl group being optionally substituted as described herein. Examples of such heterocyclic elements include, but are not limited to, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolyl, indolinyl, indolyl, isochromanyl, isoindolinyl, isoquinolinyl, isothiazolyl, naphthyridinyl, oxadiazolyl, pyridyl, pyrazinyl, pyrazolyl, pyridazinyl, pyrimidinyl, pyrrolyl, quinazolinyl, quinolinyl, quinoxalinyl, tetrahydroisoquinolinyl, tetrahydroquinolinyl, thiazolyl, thienofuryl, thienothienyl, thienyl and triazolyl. Additional nitrogen atoms may be present together with the first nitrogen and oxygen or sulfur, giving, e.g., thiadiazole.

In addition, the compounds disclosed herein may exist as tautomers and both tautomeric forms are intended to be encompassed by the scope of the invention, even though only one tautomeric structure is depicted. For example, any claim to compound A below is understood to include tautomeric structure B, and vice versa, as well as mixtures thereof.

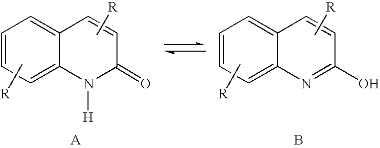

This invention is also concerned with compositions and methods of treating ocular hypertension or glaucoma by administering to a patient in need thereof one of the compounds of formula I in combination with one or more of a β-adrenergic blocking agent such as timolol, betaxolol, levobetaxolol, carteolol, levobunolol, a parasympathomimetic agent such as epinephrine, iopidine, brimonidine, clonidine, para-aminoclonidine, carbonic anhydrase inhibitor such as dorzolamide, acetazolamide, metazolamide or brinzolamide, an EP4 agonist (such as those disclosed in WO 02/24647, WO 02/42268, EP 1114816, WO 01/46140 and WO 01/72268), a prostaglandin such as latanoprost, travaprost, unoprostone, rescula, S1033 (compounds set forth in U.S. Pat. Nos. 5,889,052; 5,296,504; 5,422,368; and 5,151,444); a hypotensive lipid such as lumigan and the compounds set forth in U.S. Pat. No. 5,352,708; a neuroprotectant disclosed in U.S. Pat. No. 4,690,931, particularly eliprodil and R-eliprodil as set forth in WO 94/13275, including memantine; or an agonist of 5-HT2 receptors as set forth in PCT/US00/31247, particularly 1-(2-aminopropyl)-3-methyl-1H-imdazol-6-ol fumarate and 2-(3-chloro-6-methoxy-indazol-1-yl)-1-methyl-ethylamine. An example of a hypotensive lipid (the carboxylic acid group on the α-chain link of the basic prostaglandin structure is replaced with electrochemically neutral substituents) is that in which the carboxylic acid group is replaced with a C₁₋₆ alkoxy group such as OCH₃ (PGF₂ₐ 1-OCH₃), or a ammalia group (PGF₂ₐ 1-OH).

Preferred potassium channel blockers are calcium activated potassium channel blockers. More preferred potassium channel blockers are high conductance, calcium activated potassium (Maxi-K) channel blockers. Maxi-K channels are a family of ion channels that are prevalent in neuronal, smooth muscle and epithelial tissues and which are gated by membrane potential and intracellular Ca²⁺.

The present invention is based upon the finding that maxi-K channels, if blocked, inhibit aqueous humor production by inhibiting net solute and H₂O efflux and therefore lower IOP. This finding suggests that maxi-K channel blockers are useful for treating other ophthamological dysfunctions such as macular edema and macular degeneration. It is known that lowering IOP promotes blood flow to the retina and optic nerve. Accordingly, the compounds of this invention are useful for treating macular edema and/or macular degeneration.

It is believed that maxi-K channel blockers which lower IOP are useful for providing a neuroprotective effect. They are also believed to be effective for increasing retinal and optic nerve head blood velocity and increasing retinal and optic nerve oxygen by lowering IOP, which when coupled together benefits optic nerve health. As a result, this invention further relates to a method for increasing retinal and optic nerve head blood velocity, increasing retinal and optic nerve oxygen tension as well as providing a neuroprotective effect or a combination thereof.

A number of marketed drugs function as potassium channel antagonists. The most important of these include the compounds Glyburide, Glipizide and Tolbutamide. These potassium channel antagonists are useful as antidiabetic agents. The compounds of this invention may be combined with one or more of these compounds to treat diabetes.

Potassium channel antagonists are also utilized as Class 3 antiarrhythmic agents and to treat acute infarctions in humans. A number of naturally ammalian toxins are known to block potassium channels including Apamin, Iberiotoxin, Charybdotoxin, Noxiustoxin, Kaliotoxin, Dendrotoxin(s), mast cell degranuating (MCD) peptide, and β-Bungarotoxin (β-BTX). The compounds of this invention may be combined with one or more of these compounds to treat arrhythmias.

Depression is related to a decrease in neurotransmitter release. Current treatments of depression include blockers of neurotransmitter uptake, and inhibitors of enzymes involved in neurotransmitter degradation which act to prolong the lifetime of neurotransmitters.

Alzheimer's disease is also characterized by a diminished neurotransmitter release. Three classes of drugs are being investigated for the treatment of Alzheimer's disease cholinergic potentiators such as the anticholinesterase drugs (e.g., physostigmine (eserine), and Tacrine (tetrahydroaminocridine)); nootropics that affect neuron metabolism with little effect elsewhere (e.g., Piracetam, Oxiracetam; and those drugs that affect brain vasculature such as a mixture of ergoloid mesylates and calcium channel blocking drugs including Nimodipine. Selegiline, a monoamine oxidase B inhibitor which increases brain dopamine and norepinephrine has reportedly caused mild improvement in some Alzheimer's patients. Aluminum chelating agents have been of interest to those who believe Alzheimer's disease is due to aluminum toxicity. Drugs that affect behavior, including neuroleptics, and anxiolytics have been employed. Anxiolytics, which are mild tranquilizers, are less effective than neuroleptics The present invention is related to novel compounds which are useful as potassium channel antagonists.

The compounds within the scope of the present invention exhibit potassium channel antagonist activity and thus are useful in disorders associated with potassium channel malfunction. A number of cognitive disorders such as Alzheimer's Disease, memory loss or depression may benefit from enhanced release of neurotransmitters such as serotonin, dopamine or acetylcholine and the like. Blockage of Maxi-K channels maintains cellular depolarization and therefore enhances secretion of these vital neurotransmitters.

The compounds of this invention may be combined with anticholinesterase drugs such as physostigmine (eserine) and Tacrine (tetrahydroaminocridine), nootropics such as Piracetam, Oxiracetam, ergoloid mesylates, selective calcium channel blockers such as Nimodipine, or monoamine oxidase B inhibitors such as Selegiline, in the treatment of Alzheimer's disease. The compounds of this invention may also be combined with Apamin, Iberiotoxin, Charybdotoxin, Noxiustoxin, Kaliotoxin, Dendrotoxin(s), mast cell degranuating (MCD) peptide, β-Bungarotoxin (β-BTX) or a combination thereof in treating arrythmias. The compounds of this invention may further be combined with Glyburide, Glipizide, Tolbutamide or a combination thereof to treat diabetes.

The herein examples illustrate but do not limit the claimed invention. Each of the claimed compounds are potassium channel antagonists and are thus useful in the described neurological disorders in which it is desirable to maintain the cell in a depolarized state to achieve maximal neurotransmitter release. The compounds produced in the present invention are readily combined with suitable and known pharmaceutically acceptable excipients to produce compositions which may be administered to mammals, including humans, to achieve effective potassium channel blockage.

For use in medicine, the salts of the compounds of formula I will be pharmaceutically acceptable salts. Other salts may, however, be useful in the preparation of the compounds according to the invention or of their pharmaceutically acceptable salts. When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

The preparation of the pharmaceutically acceptable salts described above and other typical pharmaceutically acceptable salts is more fully described by Berg et al., “Pharmaceutical Salts,” J. Pharm. Sci., 1977:66:1-19.

As used herein, the term “composition” is intended to encompass a product comprising the specified ingredients in the specific amounts, as well as any product which results, directly or indirectly, from combination of the specific ingredients in the specified amounts.

When a compound according to this invention is administered into a human subject, the daily dosage will normally be determined by the prescribing physician with the dosage generally varying according to the age, weight, sex and response of the individual patient, as well as the severity of the patient's symptoms.

The maxi-K channel blockers used can be administered in a therapeutically effective amount intravaneously, subcutaneously, topically, transdermally, parenterally or any other method known to those skilled in the art.

Ophthalmic pharmaceutical compositions are preferably adapted for topical administration to the eye in the forth of solutions, suspensions, ointments, creams or as a solid insert. Ophthalmic formulations of this compound may contain from 0.01 ppm to 1% and especially 0.1 ppm to 1% of medicament. Higher dosages as, for example, about 10% or lower dosages can be employed provided the dose is effective in reducing intraocular pressure, treating glaucoma, increasing blood flow velocity or oxygen tension. For a single dose, from between 1 ng to 500 ug, preferably 1 ng to 500 ug, of the compound can be applied to the human eye.

The pharmaceutical preparation which contains the compound may be conveniently admixed with a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, polyalkylene glycols, petroleum based jelly, ethyl cellulose, ethyl oleate, carboxymethyl-cellulose, polyvinylpyrrolidone, isopropyl myristate and other conventionally employed acceptable carriers. The pharmaceutical preparation may also contain non-toxic auxiliary substances such as emulsifying, preserving, wetting agents, bodying agents and the like, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic acid, and the like. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehicles and the like. The pharmaceutical preparation may also be in the form of a microparticle formulation. The pharmaceutical preparation may also be in the form of a solid insert. For example, one may use a solid water soluble polymer as the carrier for the medicament. The polymer used to form the insert may be any water soluble non-toxic polymer, for example, cellulose derivatives such as methylcellulose, sodium carboxymethyl cellulose, (hydroxyloweralkyl cellulose), hydroxyethyl cellulose, hydroxypropyl cellulose, hydroxypropylmethyl cellulose; acrylates such as polyacrylic acid salts, ethylacrylates, polyactylamides; natural products such as gelatin, alginates, pectins, tragacanth, karaya, chondrus, agar, acacia; the starch derivatives such as starch acetate, hydroxymethyl starch ethers, hydroxypropyl starch, as well as other synthetic derivatives such as polyvinyl alcohol, polyvinyl pyrrolidone, polyvinyl methyl ether, polyethylene oxide, neutralized carbopol and xanthan gum, gellan gum, and mixtures of said polymer.

Suitable subjects for the administration of the formulation of the present invention include primates, man and other animals, particularly man and domesticated animals such as cats and dogs.

The pharmaceutical preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

The ophthalmic solution or suspension may be administered as often as necessary to maintain an acceptable IOP level in the eye. It is contemplated that administration to the ammalian eye will be about once or twice daily.

For topical ocular administration the novel formulations of this invention may take the form of solutions, gels, ointments, suspensions or solid inserts, formulated so that a unit dosage comprises a therapeutically effective amount of the active component or some multiple thereof in the case of a combination therapy.

Definitions of the terms used in the examples are as follows:

SM—Starting material,
DMSO—dimethyl sulfoxide,
TLC—thin layer chromatography,
SGC—silica gel chromatography,
PhMgBr—phenylmagnesiumbromide
h=hr=hour,
THF—tetrahydrofuran,
DMF—dimethylformamide,
min—minute,
LC/MS—liquid chromatography/mass spectrometry,
HPLC—high performance liquid chromatography,
BOP—Benzotriazol-1-yloxytris-(dimethylamino)phosphonium hexafluorophosphate,
PyBOP—Benzotriazol-1-yloxytris-pyrrolidino-phosphonium hexafluorophosphate,
equiv=eq=equivalent,

### NBS—N-Bromosuccinamide,

AIBN—2,2′-azobisisobutyronitrile,

### DCM—Dichloromethane,

mCPBA—meta-Chloroperbenzoic acid,
TFA—Trifluoroacetic acid,

### DIEA—N,N-Diisopropylethylamine,

HOBt—1-Hydroxybenzotriazole hydrate,
EDC—N-(3-Dimethylaminopropyl)-N′-ethylcarbodiimide hydrochloride,
Ph—phenyl, and
HOAt-1-Hydroxy-7-azabenzotriazole.

The following examples given by way of illustration are demonstrative of the present invention.

The 1,3,7-trisubstituted naphthalene compounds of the invention were prepared using the sequence shown in Scheme 1. The starting compound A was prepared from 7-methoxytetralone using the method of Silverman, et al. (J. Org. Chem. 50 (26), 5550, 1985). Acylation gave the desired isomer as illustrated using benzoyl chloride. Alkyl acid chloride can be used similarly.

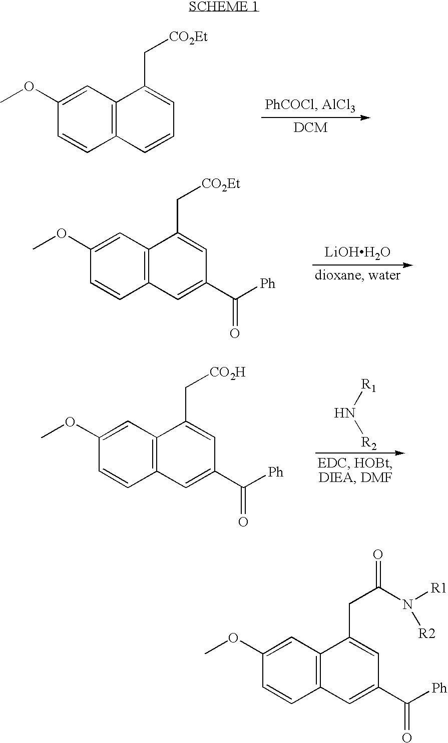

The 1,4,7-trisubstituted naphthalene isomers were prepared using the method shown in Scheme 2. The starting material in Scheme 2 was prepared in two steps from 7-methoxytetralone using the method of Hulme et al. (J. Org. Chem. 60(5), 1265, 1995). After protecting the phenol with triethylsilyl group, Friedel-Crafts reaction with excess benzoyl chloride and aluminum chloride provided the 4-benzoyl substitution. Functional group manipulations converted the 1-hydroxyl to 1-acetic acid, which was then converted to amides using standard procedures.

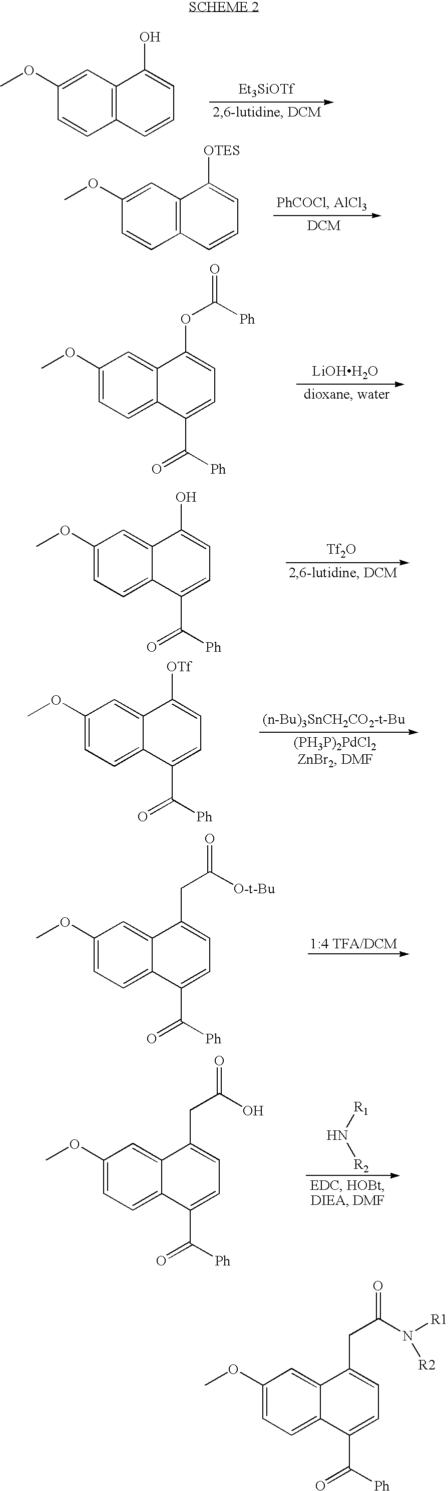

The 1,2,7-trisubstituted naphthalene isomers were prepared using the method shown in Scheme 3. A similar procedure can be used to prepare alternative 1,2,6-trisubstituted naphthalene isomers as shown in Scheme 4.

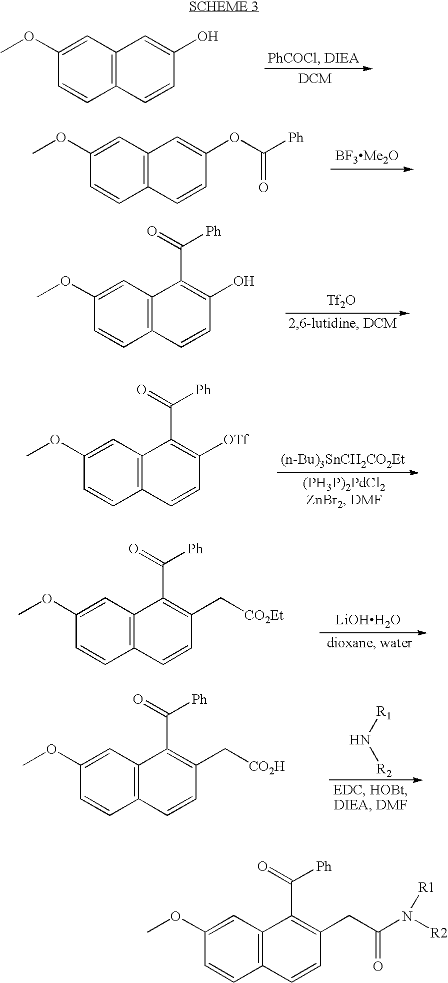

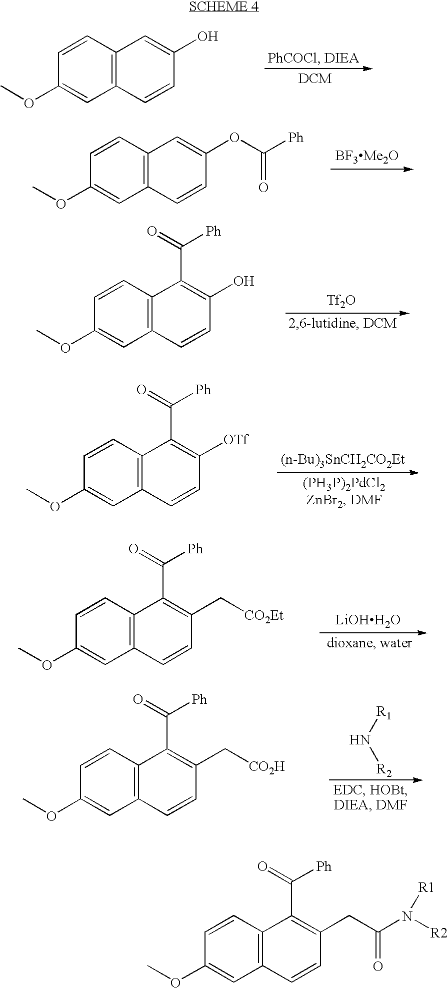

Two isomeric sets of naphthalenes were prepared using the same methods as in Schemes 3 and 4 (see Schemes 5 and 6).

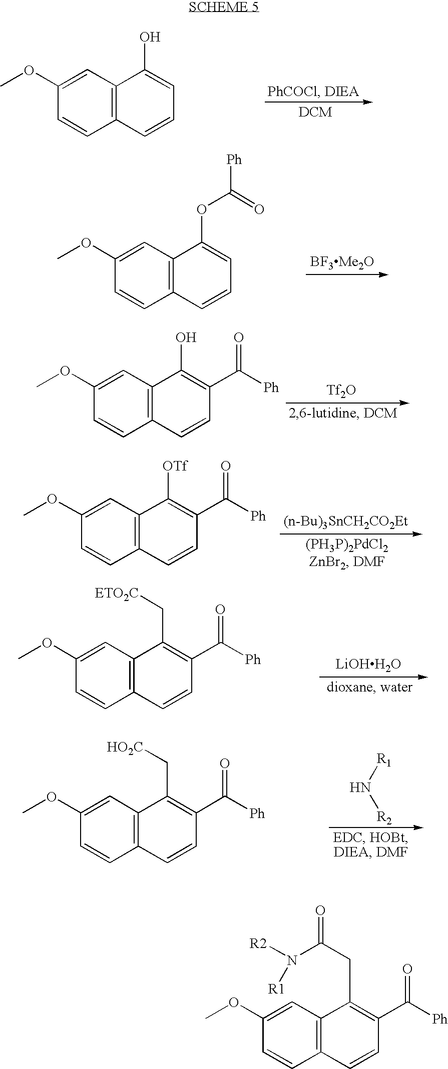

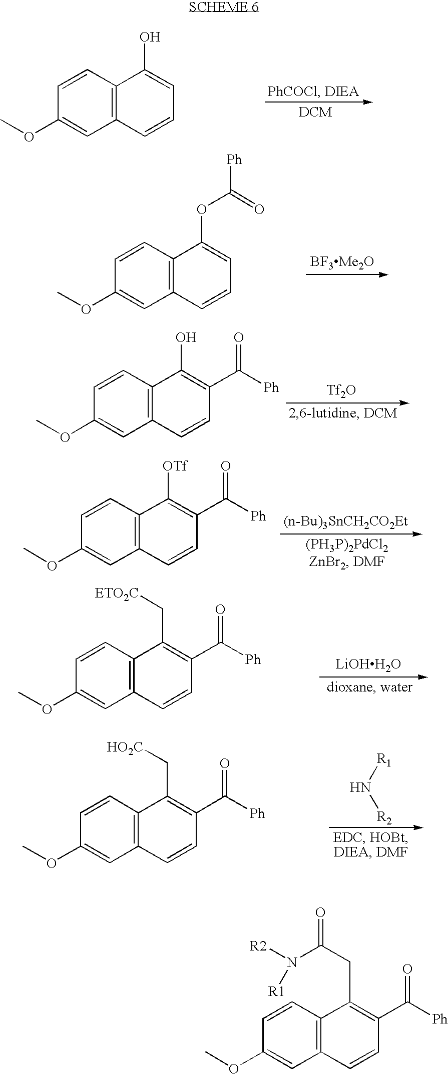

Scheme 7 illustrates the synthesis of 1,3,6-trisubstituted naphthalenes.

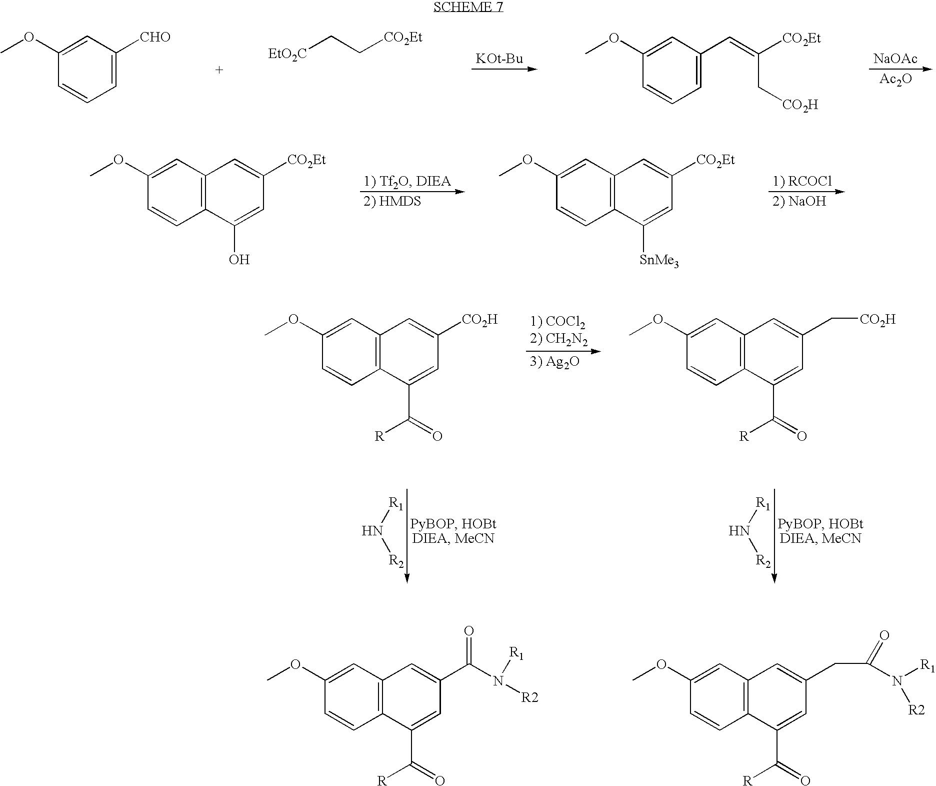

### Example 1

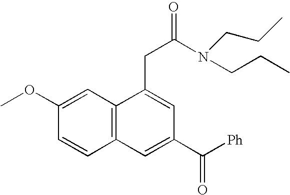

### 2-(3-Benzoyl-7-methoxy-1-naphthyl)-N,N-dipropylacetamide

Step A. Ethyl (3-benzoyl-7-methoxy-1-naphthyl)acetate

To a mixture of 1 g ethyl (7-methoxy-1-naphthyl)acetate prepared by the method of Silverman et al. (J. Org. Chem. 50, 5550, 1985) and 2.7 g anhydrous AMCl₃ in 50 mL anhydro DCM at 0° C. was added 2.0 g of benzoyl chloride. The reaction mixture was allowed to warn to room temperature overnight, quenched with ice, and worked-up with aqueous ether. The crude product was purified on SGC with 5:1 hexanes and ether to give the title compound as white solid. ¹H NMR (CDCl₃, 500 MHz) δ: 8.17 (s, 1H), 7.94 (d, 3.0 Hz, 1H), 7.86 (m, 3H), 7.63 (m, 1H), 7.53 (t, 8 Hz, 2H), 7.35 (d, 2.5 Hz, 1H), 7.23 (dd, 8.5 & 2.0 Hz, 1H), 4.19 (q, 7.5 Hz, 2H), 4.09 (s, 2H), 3.99 (s, 3H), 1.26 (t, 7.0 Hz, 3H).

Step B. (3-Benzoyl-7-methoxy-1-naphthyl)acetic acid

A mixture of 1 g ethyl (3-benzoyl-7-methoxy-1-naphthyl)acetate and 361 mg lithium hydroxide hydrate in 10 mL 1:1 dioxane and water was stirred at room temperature for 3 hours, acidified with HCl to pH ˜2, and filtered to afford the title compound after washing with ice water and drying. ¹H NMR (DMSO-d₆, 500 MHz) δ: 8.17 (s, 1H), 7.94 (d, 3.0 Hz, 1H), 7.85 (m, 3H), 7.63 (m, 1H), 7.53 (t, 8 Hz, 2H), 7.30 (m, 1H), 7.23 (dd, 8.5 & 2.0 Hz, 1H), 4.11 (s, 2H), 3.94 (s, 3H).

Step C. (3-Benzoyl-7-methoxy-1-naphthyl)-NV-dipropylacetamide

The title compound was prepared from 20 mg (3-benzoyl-7-methoxy-1-naphthyl)acetic acid, 13 μL di-n-propylamine, 18 mg EDC, 13 mg HOBt, and 33 μL DIEA in 2 mL DMF at room temperature and purified using preparative HPLC followed by lyophilization. LC-MS: 3.88 min. (m/Z 404.4).

### Examples 2˜9

The following compounds in Table 1 were prepared using the method described in Example 1 using (3-benzoyl-7-methoxy-1-naphthyl)acetic acid and the amine listed in the Table.

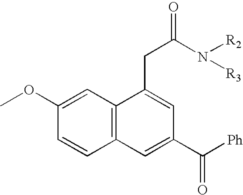

TABLE 1

| Example | Starting amine (HNR₂R₃) | LC-MS, min. (m/Z) |
|---|---|---|
| 2 | 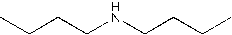 | 4.17 (432.4) |
| 3 | 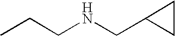 | 3.89 (416.4) |
| 4 | 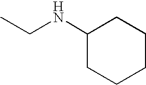 | 4.04 (430.4) |
| 5 | 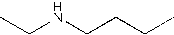 | 3.88 (404.4) |
| 6 | 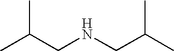 | 4.13 (432.4) |
| 7 | 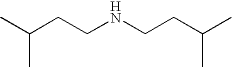 | 4.46 (460.3) |
| 8 | 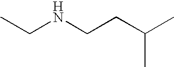 | 4.03 (418.4) |
| 9 | 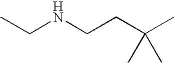 | 4.25 (432.3) |

### Example 10

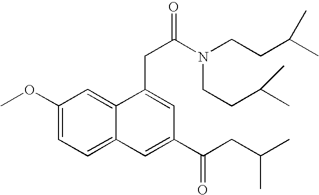

### 2-[7-Methoxy-3-(3-methylbutanoyl)-1-naphthyl]-N,N-bis(3-methylbutyl)acetamide

Step A. Ethyl[7-methoxy-3-(3-methylbutanoyl)-1-naphthyl]acetate

The title compound was prepared from ethyl (7-methoxy-1-naphthyl)acetate and 3-methylbutanoyl chloride using the method in Example 1 Step A. ¹H NMR (CDCl₃, 500 MHz) δ: 8.35 (s, 1H), 7.98 (d, 1.0 Hz, 1H), 7.90 (d, 8.5 Hz, 1H), 7.36 (d, 2.0 Hz, 1H), 7.24 (dd, 9.0 & 2.0 Hz, 1H), 4.17 (q, 7.5 Hz, 2H), 4.07 (s, 2H), 3.98 (s, 3H), 2.96 (d, 6.5 Hz, 2H), 2.83 (m, 1H), 1.25 (t, 7.0 Hz, 3H), 1.05 (d, 7.0 Hz, 6H).

Step B. [7-Methoxy-3-(3-methylbutanoyl)-1-naphthyl]acetic acid

The title compound was prepared from ethyl[7-methoxy-3-(3-methylbutanoyl)-1-naphthyl]acetate using the method in Example 1 Step B. ¹H NMR (DMSO-d₆, 500 MHz) δ: 8.52 (s, 1H), 8.07 (d, 9.0 Hz, 1H), 7.87 (d, 1.5 Hz, 1H), 7.33 (d, 2.0 Hz, 1H), 7.27 (dd, 9.0 & 2.0 Hz, 1H), 4.05 (s, 2H), 3.89 (s, 3H), 2.97 (d, 7.0 Hz, 2H), 2.21 (m, 1H), 0.95 (d, 7.0 Hz, 6H).

Step C. 2-[7-Methoxy-3-(3-methylbutanoyl)-1-naphthyl]-N,N-bis(3-methylbutyl)acetamide

The title compound was prepared from [7-methoxy-3-(3-methylbutanoyl)-1-naphthyl]acetic acid and 3-methyl-N-(3-methylbutyl)butan-1-amine using the method in Example 1 Step C. LC-MS, 4.59 min. (m/Z 440.4)

### Example 11

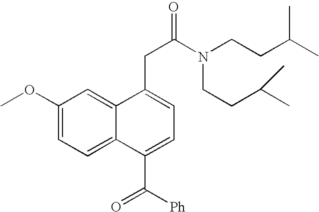

### 2-(4-Benzoyl-7-methoxy-1-naphthyl)-N,N-bis(3-methylbutyl)acetamide

Step A. 7-Methoxy-1-naphthol

The title compound was prepared from 7-methoxytetralone using the method of Hulme et al. (J. Org. Chem. 60(5), 1265, 1995). ¹H NMR (CDCl₃, 500 MHz) δ: 7.74 (d, 9.5 Hz, 1H), 7.50 (d, 2.5 Hz, 1H), 7.41 (d, 8.5 Hz, 1H), 7.21-7.18 (m, 2H), 6.82 (d, 7.0 Hz, 1H), 3.98 (s, 3H).

Step B. tert-Butyl[(7-methoxy-1-naphthyl)oxy]dimethylsilane

To a solution of 25 g 7-methoxy-1-naphthol in 500 mL DCM at 0° C. was added 26.14 g 2,6-diemthylpyridine followed by 45.52 g tert-butyl(dimethyl)silyl trifluoromethanesulfonate. The mixture was allowed to warm up to room temperature overnight and worked up with water and DCM. The crude product was purified with SGC using hexanes to give the title compound. ¹H NMR (CDCl₃, 500 MHz) δ: 7.73 (d, 8.5 Hz, 1H), 7.53 (d, 2.5 Hz, 1H), 7.42 (d, 8.0 Hz, 1H), 7.21 (t, 8.0 Hz, 1H), 7.16 (dd, 9.0 & 2.5 Hz, 1H), 6.88 (d, 7.5 Hz, 1H), 3.95 (s, 3H), 1.15 (s, 9H), 0.31 (s, 6H).

Step C. 4-Benzoyl-7-methoxy-1-naphthyl benzoate

The title compound was prepared from tert-butyl[(7-methoxy-1-naphthyl)oxy]dimethylsilane with 3 equiv. of benzoyl chloride and 5 equiv. of anhydrous aluminum chloride using the method described in Example 1 Step A. ¹H NMR (CDCl₃, 500 MHz) δ: 8.39 (d, 7.0 Hz, 2H), 8.16 (d, 9.5 Hz, 1H), 7.93 (d, 7.0 Hz, 2H), 7.74 (m, 1H), 7.63-7.61 (m, 3H), 7.54-7.49 (m, 3H), 7.42 (d, 8.0 Hz, 1H), 7.33 (d, 2.5 Hz, 1H), 7.25 (dd, 9.0 & 2.5 Hz, 1H), 3.88 (s, 3H).

Step D. (4-Hydroxy-6-methoxy-1-naphthyl)(phenyl)methanone

The title compound was prepared from 4-benzoyl-7-methoxy-1-naphthyl benzoate using the method described in Example 1 Step B. ¹H NMR (CDCl₃, 500 MHz) δ: 8.31 (d, 9.5 Hz, 1H), 7.85 (m, 2H), 7.62-7.57 (m, 2H), 7.50-7.46 (m, 2H), 7.37 (d, 8.0 Hz, 1H), 7.25 (dd, 9.0 & 2.5 Hz, 1H), 6.77 (d, 8.0 Hz, 1H), 3.97 (s, 3H).

Step E. 4-Benzoyl-7-methoxy-1-naphthyl trifluoromethanesulfonate

The title compound was prepared from (4-hydroxy-6-methoxy-1-naphthyl)(phenyl)methanone and 1.3 equiv. triflic anhydride in the presence of 1.5 equiv. of 2,6-lutidine in DCM at 0° C. in one hour. ¹H NMR (CDCl₃, 500 MHz) δ: 8.02 (d, 9.5 Hz, 1H), 7.88-7.86 (m, 2H), 7.66 (m, 1H), 7.52-7.49 (m, 3H), 7.46-7.42 (m, 2H), 7.27 (dd, 9.0 & 2.5 Hz, 1H), 4.00 (s, 3H).

Step F. tert-Butyl (4-benzoyl-7-methoxy-1-naphthyl)acetate

The title compound was prepared from 4.1 g 4-benzoyl-7-methoxy-1-naphthyl trifluoromethanesulfonate, 4.5 g tert-butyl (tributylstannyl)acetate (prepared using the method of Zapata et al. from Syn. Commun. 1984 (14), 27), 350 mg of (Ph₃P)₂PdCl₂, and 2.3 g zinc bromide in 50 mL DMF at 90° C. for 12 hours. After removal of solvent under reduced pressure, aqueous work-up using EtOAc and SGC using toluene having 0˜20% ether provided pure title compound. ¹H NMR (CDCl₃, 500 MHz) δ: 8.07 (d, 9.0 Hz, 1H), 7.89-7.87 (m, 2H), 7.61 (m, 1H), 7.47 (t, 7.5 Hz, 2H), 7.44-7.40 (m, 2H), 7.37 (d, 2.5 Hz, 1H), 7.19 (dd, 9.0 & 2.5 Hz, 1H), 4.01 (s, 2H), 3.98 (s, 3H), 1.47 (s, 9H).

Step G. (4-Benzoyl-7-methoxy-1-naphthyl)acetic acid

The title compound was prepared from tert-butyl (4-benzoyl-7-methoxy-1-naphthyl)acetate in 1:4 TFA and DCM. ¹H NMR (CD₃OD, 500 MHz) δ: 7.93 (d, 9.0 Hz, 1H), 7.83-7.81 (m, 2H), 7.64 (m, 1H), 7.51-7.47 (m, 4H), 7.35 (d, 7.5 Hz, 1H), 7.16 (dd, 9.5 & 2.0 Hz, 1H), 4.10 (s, 2H), 3.94 (s, 3H).

Step H. 2-(4-Benzoyl-7-methoxy-1-naphthyl)-N,N-bis(3-methylbutyl)acetamide

The title compound was prepared from (4-benzoyl-7-methoxy-1-naphthyl)acetic acid and 3-methyl-N-(3-methylbutyl)butan-1-amine using the method in Example 1 Step C. LC-MS, 4.51 min. (m/Z 460.3)

### Example 12

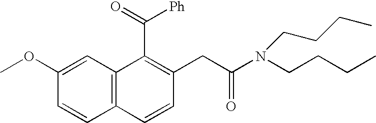

### 2-(1-Benzoyl-7-methoxy-2-naphthyl)-N,N-dibutylacetamide

Step A. 7-Methoxy-2-naphthyl benzoate

The title compound was prepared from 25 g 7-methoxy-2-naphthol, 22.2 g benzoyl chloride and 27.8 g DIEA in 200 mL DCM at 0° C. for 10 minutes and at room temperature for 2 hours. The crude product from aqueous work-up was purified with SGC using 2:1 toluene and hexanes to afford the title compound. ¹H NMR (CDCl₃, 500 MHz) δ: 8.30-8.29 (m, 2H), 7.75 (d, 9.0 Hz, 1H), 7.79 (d, 9.0 Hz, 1H), 7.69 (m, 1H), 7.63 (d, 2.5 Hz, 1H), 7.57 (m, 2H), 7.25 (dd, 9.0 & 2.0 Hz, 1H), 7.18 (dd, 9.0 & 2.5 Hz, 1H), 7.16 (d, 2.5 Hz, 1H), 3.95 (s, 3H).

Step B. (2-hydroxy-7-methoxy-1-naphthyl)(phenyl)methanone

The title compound was prepared by refluxing 8 g of 7-methoxy-2-naphthyl benzoate in 35 mL boron trifluoride methyl etherate for 1 hour, followed by aqueous work-up with DCM and SGC using hexanes and ether (100:6). ¹H NMR (CDCl₃, 500 MHz) δ: 7.88 (d, 9.0 Hz, 1H), 7.66-7.64 (m, 3H), 7.58 (m, 1H), 7.48-7.46 (m, 2H), 7.11 (d, 8.5 Hz, 1H), 6.92 (dd, 9.0 & 2.5 Hz, 1H), 7.62 (d, 2.5 Hz, 1H), 3.30 (s, 3H).

Step C. 1-Benzoyl-7-methoxy-2-naphthyl trifluoromethanesulfonate

The title compound was prepared from (2-hydroxy-7-methoxy-1-naphthyl)phenyl)methanone using method described for Example 11 Step E. ¹H NMR (CDCl₃, 500 MHz) δ: 7.99 (d, 9.0 Hz, 1H), 7.88-7.85 (m, 3H), 7.66 (m, 1H), 7.51-7.48 (m, 2H), 7.39 (d, 9.0 Hz, 1H), 7.25 (dd, 9.0 & 2.5 Hz, 1H), 6.93 (d, 2.5 Hz, 1H), 3.74 (s, 3H).

Step D. Ethyl (1-benzoyl-7-methoxy-2-naphthyl)acetate

The title compound was prepared from 1-benzoyl-7-methoxy-2-naphthyl trifluoromethanesulfonate and ethyl (tributylstannyl)acetate using method described in Example 11 Step F. ¹H NMR (CDCl₃, 500 MHz) δ: 7.88-7.84 (m, 3H), 7.80 (d, 9.0 Hz, 1H), 7.60 (m, 1H), 7.46-7.43 (m, 2H), 7.38 (d, 9.0 Hz, 1H), 7.15 (dd, 9.0 & 2.5 Hz, 1H), 6.77 (d, 2-5 Hz, 1H), 3.98 (q, 7.0 Hz, 2H), 3.66 (s, 2H), 3.65 (s, 3H), 1.10 (t, 7.0 Hz, 3H).

Step E. (1-Benzoyl-7-methoxy-2-naphthyl)acetic acid

The title compound was prepared from ethyl (1-benzoyl-7-methoxy-2-naphthyl)acetate using the method described in Example 1 Step B. ¹H NMR (DMSO-d₆, 500 MHz) δ: 7.91 (m, 2H), 7.71 (d, 7.0 Hz, 2H), 7.64 (m, 1H), 7.48 (t, 7.5 Hz, 2H), 7.40 (d, 8.0 Hz, 1H), 7.17 (dd, 9.0 & 2.5 Hz, 1H), 6.57 (d, 2.5 Hz, 1H), 3.56 (s, 3H), 3.44 (s, 2H).

Step F. 2-(1-Benzoyl-7-methoxy-2-naphthyl)-N,N-dibutylacetamide

The title compound was prepared from (1-benzoyl-7-methoxy-2-naphthyl)acetic acid and dibutylamine using the method in Example 1 Step C. LC-MS, 4.28 min. (m/Z 432.3)

### Example 13

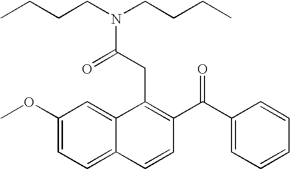

### 2-(2-Benzoyl-7-methoxy-1-naphthyl)-N,N-dibutylacetamide

Steps A˜E. (2-Benzoyl-7-methoxy-1-naphthyl)acetic acid

The title compound was prepared from 7-methoxy-1-naphthol using the same sequence as described in Example 12 Steps A˜E. ¹H NMR (DMSO-d₆, 500 MHz) δ: 7.95 (d, 9.0 Hz, 1H), 7.71 (d, 8.5 Hz, 1H), 7.73-7.71 (m, 2H), 7.67 (m, 1H), 7.53 (t, 8.0 Hz, 2H), 7.42 (d, 2.5 Hz, 1H), 7.31 (dd, 9.0 & 2.5 Hz, 1H), 7.23 (d, 7.5 Hz, 1H), 4.04 (s, 2H), 3.90 (s, 3H).

Steps F. 2-(2-Benzoyl-7-methoxy-1-naphthyl)-N,N-dibutylacetamide

The title compound was prepared from (2-benzoyl-7-methoxy-1-naphthyl)acetic acid and dibutylamine using the method in Example 1 Step C. LC-MS, 4.33 min. (m/Z 432.0)

### Example 14

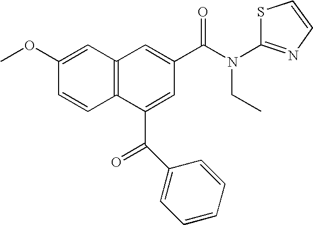

### 4-Benzoyl-N-ethyl-7-methoxy-N-1,3-thiazol-2-yl-2-naphthamide

The title compound was prepared from 42 mg 4-benzoyl-7-methoxy-2-naphthoic acid, 60 mg N-ethyl-1,3-thiazol-2-amine, 142 mg PyBOP, and 83 μL DIEA in 2 mL MeCN by heating at 65 and 100C for one hour each followed by RP-HPLC purification.

### Example 15

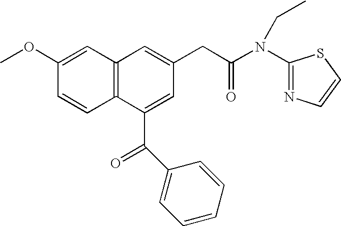

### 2-(4-Benzoyl-7-methoxy-2-naphthyl)-N-ethyl-N-1,3-thiazol-2-ylacetamide

The title compound was prepared from 18 mg (4-benzoyl-7-methoxy-2-naphthyl)acetic acid, 30 mg N-ethyl-1,3-thiazol-2-amine, 35 mg BOP, and 31 μL DIEA in 1 mL MeCN by heating at 85C for one hour followed by RP-HPLC purification.

### Functional Assays

A. Maxi-K Channel

The activity of the compounds can also be quantified by the following assay.

The identification of inhibitors of the Maxi-K channel is based on the ability of expressed Maxi-K channels to set cellular resting potential after transfection of both alpha and beta1 subunits of the channel in HEK-293 cells and after being incubated with potassium channel blockers that selectively eliminate the endogenous potassium conductances of HEK-293 cells. In the absence of maxi-K channel inhibitors, the transfected HEK-293 cells display a hyperpolarized membrane potential, negative inside, close to E_{K} (−80 mV) which is a consequence of the activity of the maxi-K channel. Blockade of the Maxi-K channel by incubation with maxi-K channel blockers will cause cell depolarization. Changes in membrane potential can be determined with voltage-sensitive fluorescence resonance energy transfer (FRET) dye pairs that use two components, a donor coumarin (CC₂DMPE) and an acceptor oxanol (DiSBAC₂(3)).

Oxanol is a lipophilic anion and distributes across the membrane according to membrane potential. Under normal conditions, when the inside of the cell is negative with respect to the outside, oxanol is accumulated at the outer leaflet of the membrane and excitation of coumarin will cause FRET to occur. Conditions that lead to membrane depolarization will cause the oxanol to redistribute to the inside of the cell, and, as a consequence, to a decrease in FRET. Thus, the ratio change (donor/acceptor) increases after membrane depolarization, which determines if a test compound actively blocks the maxi-K channel.

The HEK-293 cells were obtained from the American Type Culture Collection, 12301 Parklawn Drive, Rockville, Md., 20852 under accession number ATCC CRL-1573. Any restrictions relating to public access to the microorganism shall be irrevocably removed upon patent issuance.

Transfection of the alpha and beta1 subunits of the maxi-K channel in HEK-293 cells was carried out as follows: HEK-293 cells were plated in 100 mm tissue culture treated dishes at a density of 3×10⁶ cells per dish, and a total of five dishes were prepared. Cells were grown in a medium consisting of Dulbecco's Modified Eagle Medium (DMEM) supplemented with 10% Fetal Bovine serum, 1× L-Glutamine, and 1× Penicillin/Streptomycin, at 37° C., 10% CO₂. For transfection with Maxi-K hα(pCIneo) and Maxi-K hβ1 (pIRESpuro) DNAs, 150 μl FuGENE6™ was added dropwise into 10 ml of serum free/phenol-red free DMEM and allowed to incubate at room temperature for 5 minutes. Then, the FuGENE6™ solution was added dropwise to a DNA solution containing 25 μg of each plasmid DNA, and incubated at room temperature for 30 minutes. After the incubation period, 2 ml of the FuGENE6™/DNA solution was added dropwise to each plate of cells and the cells were allowed to grow two days under the same conditions as described above. At the end of the second day, cells were put under selection media which consisted of DMEM supplemented with both 600 μg/ml G418 and 0.75 μg/ml puromycin. Cells were grown until separate colonies were formed. Five colonies were collected and transferred to a 6 well tissue culture treated dish. A total of 75 colonies were collected. Cells were allowed to grow until a confluent monolayer was obtained. Cells were then tested for the presence of maxi-K channel alpha and beta1 subunits using an assay that monitors binding of ¹²⁵I-iberiotoxin-D19Y/Y36F to the channel. Cells expressing ¹²⁵I-iberiotoxin-D 19Y/Y36F binding activity were then evaluated in a functional assay that monitors the capability of maxi-K channels to control the membrane potential of transfected HEK-293 cells using fluorescence resonance energy transfer (FRET) ABS technology with a VIPR instrument. The colony giving the largest signal to noise ratio was subjected to limiting dilution. For this, cells were resuspended at approximately 5 cells/ml, and 200 pt were plated in individual wells in a 96 well tissue culture treated plate, to add ca. one cell per well. A total of two 96 well plates were made. When a confluent monolayer was formed, the cells were transferred to 6 well tissue culture treated plates. A total of 62 wells were transferred. When a confluent monolayer was obtained, cells were tested using the FRET-functional assay. Transfected cells giving the best signal to noise ratio were identified and used in subsequent functional assays.

### For Functional Assays:

The transfected cells (2E+06 Cells/mL) are then plated on 96-well poly-D-lysine plates at a density of about 100,000 cells/well and incubated for about 16 to about 24 hours. The medium is aspirated of the cells and the cells washed one time with 100 μl of Dulbecco's phosphate buffered saline (D-PBS). One hundred microliters of about 9 μM coumarin (CC₂DMPE)-0.02% pluronic-127 in D-PBS per well is added and the wells are incubated in the dark for about 30 minutes. The cells are washed two times with 100 μl of Dulbecco's phosphate-buffered saline and 100 μl of about 4.5 μM of oxanol (DiSBAC₂(3)) in (mM) 140 NaCl, 0.1 KCl, 2 CaCl₂, 1 MgCl₂, 20 Hepes-NaOH, pH 7.4, 10 glucose is added. Three micromolar of an inhibitor of endogenous potassium conductance of HEK-293 cells is added. A maxi-K channel blocker is added (about 0.01 micromolar to about 10 micromolar) and the cells are incubated at room temperature in the dark for about 30 minutes.

The plates are loaded into a voltage/ion probe reader (VIPR) instrument, and the fluorescence emission of both CC₂DMPE and DiSBAC₂(3) are recorded for 10 sec. At this point, 100 μl of high-potassium solution (mM): 140 KCl, 2 CaCl₂, 1 MgCl₂; 20 Hepes-KOH, pH 7.4, 10 glucose are added and the fluorescence emission of both dyes recorded for an additional 10 sec. The ratio CC₂DMPE/DiSBAC₂(3), before addition of high-potassium solution equals 1. In the absence of maxi-K channel inhibitor, the ratio after addition of high-potassium solution varies between 1.65-2.0. When the Maxi-K channel has been completely inhibited by either a known standard or test compound, this ratio remains at 1. It is possible, therefore, to titrate the activity of a Maxi-K channel inhibitor by monitoring the concentration-dependent change in the fluorescence ratio.

The compounds of this invention were found to cause concentration-dependent inhibition of the fluorescence ratio with IC₅₀ 's in the range of about 1 nM to about 500 μM, more preferably from about 5 nM to about 20 nM.

What is claimed is:

1. A compound of formula I: 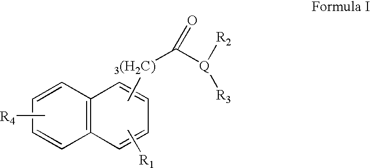
or a pharmaceutically acceptable salt, ester including phosphate, enantiomer, diastereomer or mixture thereof:
wherein,
R and R^{y} independently represent hydrogen, or C₁₋₆ alkyl;
R₁ represents hydrogen or C₁₋₆ alkyl, CF₃, (CH₂)ₙC₃₋₁₀ cycloalkyl, (CH₂)ₙC₆₋₁₀ aryl, —(CH₂)ₙC₅₋₁₀ heteroaryl, C₁₋₆ alkoxy, OH, COR^{c}, said alkyl, cycloalkyl, aryl, heteroaryl, and alkoxy optionally substituted with 1-3 groups selected from R^{b};
Q represents N, CR^{y}, or O, wherein R₂ is absent when Q is O;
R₂ represents hydrogen, C₁₋₁₀ alkyl, C₂₋₁₀ hydroxylalkyl, C₁₋₆ alkyl SR, —(CH₂)ₙO(CH₂)ₘOR, (CH₂)ₘOR, —(CH₂)ₙ(CHR₇)_{S}(CH₂)ₘC₁₋₆ alkoxy, —(CH₂)ₙ(CHR₇)_{S}(CH₂)ₘC₃₋₈ cycloalkyl, —(CH₂)ₙ(CHR₇)_{S}(CH₂)ₘC₃₋₁₀ heterocyclyl, —(CH₂)ₙC₅₋₁₀ heteroaryl, —N(R)₂, —COOR, or —(CH₂)ₙ(CHR₇)_{S}(CH₂)ₘC₆₋₁₀ aryl, said alkyl, cycloalkyl, heterocyclyl, aryl or heteroaryl optionally substituted with 1-3 groups selected from R^{a};
R₃ represents hydrogen, C₁₋₁₀ alkyl, C₂₋₆ alkenyl, —(CH₂)ₙ(CHR₇)_{S}(CH₂)ₘC₃₋₈ cycloalkyl, —(CH₂)ₙ(CHR₇)_{S}(CH₂)ₘC₃₋₁₀ heterocyclyl, —(CH₂)ₙ(CHR₇)_{S}(CH₂)ₘC₅₋₁₀ heteroaryl, —(CH₂)ₙ(CHR₇)S(CH₂)ₘCOOR, —(CH₂)ₙ(CHR₇)_{S}(CH₂)ₘC₆₋₁₀ aryl, —(CH₂)ₙ(CHR₇)_{S}(CH₂)ₘNHR₈, —(CH₂)ₙ(CHR₇)_{S}(CH₂)ₘN(R)₂, —(CH₂)ₙ(CH₇)_{S}(CH₂)ₘN(R₈)₂, —(CH₂)ₙ(CHR₇)_{S}(CH₂)ₘNHCOOR, —(CH₂)ₙ(CHR₇)_{S}(CH₂)ₘN(R₈)CO₂R, —(CH₂)ₙ(CHR₇)_{S}(CH₂)ₘN(R₈)COR, —(CH₂)ₙ(CHR₇)S(CH₂)ₘNHCOR, —(CH₂)ₙ(CHR₇)_{S}(CH₂)ₘCONH(R₈), aryl, —(CH₂)ₙ(CHR₇)_{S}(CH₂)ₘOR, —(CH₂)ₙC(R₇)₂(CH₂)ₘOR, CF₃, (CH₂)ₙ(CHR₇)_{S}(CH₂)ₘSO₂R, —(CH₂)ₙ(CHR₇)S(CH₂)ₘSO₂N(R)₂, —(CH₂)ₙ(CHR₇)_{S}(CH₂)ₘCON(R)₂, —(CH₂)ₙ(CHR₇)_{S}(CH₂)ₘCONHC(R)₃, —(CH₂)ₙCONHC(R)₂CO₂R, —(CH₂)ₙ(CHR₇)_{S}(CH₂)ₘCOR₈, nitro, cyano or halogen, said alkyl, cycloalkyl, alkoxy, heterocyclyl, aryl or heteroaryl optionally substituted with 1-3 groups of R^{a};
or, when Q is N, R₂ and R₃ taken together with the intervening N atom form a 4-10 membered heterocyclic ring optionally interrupted by 1-2 atoms of O, S, C(O) or NR, and optionally having 1-4 double bonds, and optionally substituted by 1-3 groups selected from R^{a};
or, when Q equals CR^{y}, R₂ and R₃ taken together with the intervening CR^{y} form a 4-10 membered carbocyclic or heterocyclic aromatic ring or fused ring optionally interrupted by 1-2 atoms of O, S, C(O) or NR, and optionally having 1-5 double bonds, and optionally substituted by 1-3 groups selected from R^{a},
R₄ represents hydrogen, C₁₋₆ alkoxy, halogen, cyano, OH, C₁₋₆ alkyl, COOR, SO₃H, C₁₋₆ alkylcarbonyl, S(O)_{q}R^{y}, —O(CH₂)ₙN(R)₂, —O(CH₂)ₙCO₂R, —OPO(OH)₂, CF₃, —N(R)₂, nitro, or C₁₋₆ alkylamino;
R₇ represents hydrogen, C₁₋₆ alkyl, —(CH₂)ₙCOOR or —(CH₂)ₙN(R)₂,
R₈ represents —(CH₂)ₙC₃₋₈ cycloalkyl, —(CH₂)_{n 3}-10 heterocyclyl, C₁₋₆ alkoxy or —(CH₂)ₙC₅₋₁₀ heteroaryl, —(CH₂)ₙC₆₋₁₀ aryl said heterocyclyl, cycloalkyl, aryl or heteroaryl optionally substituted with 1-3 groups selected from R^{a};
R^{a} represents F, Cl, Br, I, CF₃, N(R)₂, NO₂, CN, —(CH₂)ₙCOR₈, —(CH₂)ₙCONHR₈, —(CH₂)ₙCON(R₈)₂, —O(CH₂)ₙCOOR, —NH(CH₂)ₙOR, —COOR, —OCF₃, —O—, —NHCOR, —SO₂R, —SO₂NR₂, —SR, (C₁-C₆ alkyl)O—, —(CH₂)ₙO(CH₂)ₘOR, —(CH₂)ₙC₁₋₆ alkoxy, (aryl)O—, —OH, (C₁-C₆ alkyl)S(O)ₘ—, H₂N—CH—, (C₁-C₆ alkyl)C(O)—, (C₁-C₆ alkyl)OC(O)NH—, —(C₁-C₆ alkyl)NR_{w}(CH₂)ₙC₃₋₁₀ heterocyclyl-R_{w}, —(C₁-C₆ alkyl)O(CH₂)ₙC₃₋₁₀ heterocyclyl-R_{w}, —(C₁-C₆ alkyl)S(CH₂)ₙC₃₋₁₀ heterocyclyl-R_{w}, —(C₁-C₆ alkyl)-C₃₋₁₀ heterocyclyl-R_{w}, —(CH₂)ₙ—K—C(═K)N(R)₂, —(C₂₋₆ alkenyl)NR_{w}(CH₂)ₙC₃₋₁₀ heterocyclyl-R_{w}, —(C₂₋₆ alkenyl)O(CH₂)ₙC₃₋₁₀ heterocyclyl-R_{w}, —(C₂₋₆ alkenyl)S(CH₂)ₙC₃₋₁₀ heterocyclyl-R_{w}, —(C₂₋₆ alkenyl)-C₃₋₁₀ heterocyclyl-R_{w}, —(C₂₋₆ alkenyl)-K—C(═K)N(R)₂, —(CH₂)ₙSO₂R, —(CH₂)ₙSO₃H, —(CH₂)ₙPO(OR)₂, —(CH₂)ₙOPO(OR)₂, cyclohexyl, cyclopentyl, morpholinyl, piperidyl, pyrrolidinyl, thiophenyl, phenyl, pyridyl, imidazolyl, oxazolyl, isoxazolyl, thiazolyl, thienyl, furyl, isothiazolyl, C₂₋₆ alkenyl, and C₁-C₁₀ alkyl, said alkyl, alkenyl, alkoxy, phenyl, pyridyl, imidazolyl, oxazolyl, isoxazolyl, thiazolyl, thienyl, furyl, and isothiazolyl optionally substituted with 1-3 groups selected from C₁-C₆ alkyl, and COOR;
K independently represents CH, CH₂ or NH;
R_{w} represents H, C₁₋₆ alkyl, —C(O)C₁₋₆ alkyl, —C(O)OC₁₋₆ alkyl, —SO₂N(R)₂, —SO₂C₁₋₆ alkyl, —SO₂C₆₋₁₀ aryl, NO₂, CN or —C(O)N(R)₂;
R^{b} represents C₁₋₆ alkyl, —COOR, —SO₃R, CN, (CH₂)ₙOR, C(O)O(CH₂)ₙC(O)R, —OPO(OH)₂, —(CH₂)ₙC₆₋₁₀ aryl, or —(CH₂)ₙC₅₋₁₀ heteroaryl;
R^{c} represents hydrogen, C₁₋₆ alkyl, or —(CH₂)ₙC₆₋₁₀ aryl;
m is 0-3;
n is 0-3;
q is 0-2; and
s is 0-2.

2. A compound according to claim 1 wherein Q is N.

3. A compound according to claim 2 wherein QR₂R₃ is a dialkylamine or hydroxylamine and R₁ is —C(O)R^{c}.

4. The compound according to claim 1 of structural formula II: 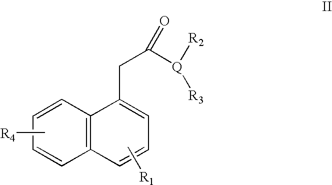
or a pharmaceutically acceptable salt, enantiomer, diastereomer or mixture thereof:
wherein Q is N.

5. The compound according to claim 1 of structural formula III: 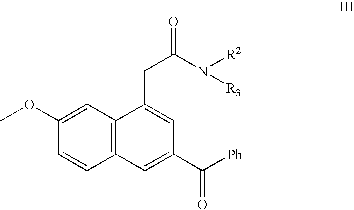
or a pharmaceutically acceptable salt, enantiomer, diastereomer or mixture thereof:
wherein R₂ and R₃ are independently C₁₋₁₀ alkyl, (CH₂)ₙC₃₋₁₀ cycloalkyl, —(CH₂)ₙ(CHR₇)_{S}(CH₂)ₘC₆₋₁₀ aryl, (CH₂)ₙ(CHR₇)_{S}(CH₂)ₘC₃₋₁₀ heterocyclyl, said alkyl, cycloalkyl, heterocyclyl, and aryl optionally substituted with 1-3 groups selected from R^{a}.

6. The compound according to claim 1 which is:
2-(3-Benzoyl-7-methoxy-1-naphthyl)-N,N-dipropylacetamide;
2-(3-Benzoyl-7-methoxy-1-naphthyl)-N,N-dibutylacetamide;
2-(3-Benzoyl-7-methoxy-1-naphthyl)-N-(cyclopropylmethyl)-N-propylacetamide;
2-(3-Benzoyl-7-methoxy-1-naphthyl)-N-(cyclohexyl)-N-ethylacetamide;
2-(3-Benzoyl-7-methoxy-1-naphthyl)-N-ethyl-N-butylacetamide;
2-(3-Benzoyl-7-methoxy-1-naphthyl)-N,N-diisobutylacetamide;
2-(3-Benzoyl-7-methoxy-1-naphthyl)-N,N-bis(3-methylbutyl)acetamide;
2-(3-Benzoyl-7-methoxy-1-naphthyl)-N-ethyl-N-(3-methylbutyl)acetamide;
2-(3-Benzoyl-7-methoxy-1-naphthyl)-N-(3,3-dimethylbutyl)-N-ethylacetamide;
2-[7-Methoxy-3-(3-methylbutanoyl)-1-naphthyl]-N,N-bis(3-methylbutyl)acetamide;
2-(4-Benzoyl-7-methoxy-1-naphthyl)-N,N-bis(3-methylbutyl)acetamide;
2-(1-Benzoyl-7-methoxy-2-naphthyl)-N,N-dibutylacetamide;
2-(2-Benzoyl-7-methoxy-1-naphthyl)-N,N-dibutylacetamide;
4-Benzoyl-N-ethyl-7-methoxy-N-1,3-thiazol-2-yl-2-naphthamide;
2-(4-Benzoyl-7-methoxy-2-naphthyl)-N-ethyl-N-1,3-thiazol-2-ylacetamide;
or a pharmaceutically acceptable salt, ester, including phosphate, enantiomer, diastereomer or mixture thereof.

7. A method for treating ocular hypertension or glaucoma comprising administration to a patient in need of such treatment a therapeutically effective amount of a compound of structural formula I of claim 1.

8. A method for treating macular edema, macular degeneration, increasing retinal and optic nerve head blood velocity, increasing retinal and optic nerve oxygen tension, and/or a neuroprotective effect comprising administration to a patient in need of such treatment a pharmaceutically effective amount of a compound of claim 1; or a pharmaceutically acceptable salt, ester including phosphate, enantiomer, diastereomer or mixture thereof.

9. A composition comprising a compound of formula I of claim 1 and a pharmaceutically acceptable carrier.

10. A composition according to claim 9 wherein one or more of an active ingredient belonging to the group consisting of: β-adrenergic blocking agent, parasympathomimetic agent, sympathomimetic agent, carbonic anhydrase inhibitor, EP4 agonist, a prostaglandin or derivative thereof, hypotensive lipid, neuroprotectant, and/or 5-HT2 receptor agonist is optionally added.
